(19) United States
(12) Patent Application Publication
(10) Pub. No.: US 2026/0090714 A1
(43) Pub. Date: Apr. 2, 2026

(54) FUNDUS IMAGING APPARATUS

(71) Applicant: NIDEK CO.,LTD., Gamagori-shi (JP)

(72) Inventors: Yusuke OOGI, Gamagori-shI (JP); Rui FUKUOKA, Gamagori-shi (JP); Daisuke BABA, Gamagori-shi (JP)

(21) Appl. No.: 19/339,331

(22) Filed: Sep. 25, 2025

(30) Foreign Application Priority Data
Sep. 30, 2024 (JP) 2024-171794

(51) Int. Cl. A61B 3/10; A61B 3/12

(52) U.S. Cl. CPC A61B 3/102; A61B 3/12

(57) ABSTRACT

A fundus imaging apparatus includes a base, a display unit, an imaging optical system, an OCT optical system, a control unit configured to execute at least (i) imaging control of an imaging optical system and an OCT optical system, (ii) display control processing of a color image and an OCT image based on OCT data, and (iii) analysis processing of the OCT data, and an imaging unit housing the imaging optical system, the OCT optical system, and the control unit therein. The control unit is disposed above the imaging optical system and the OCT optical system in the imaging unit. The imaging unit includes an exhaust port that discharges air heated by the control unit. The exhaust port is located at the same height as the control unit or at a position higher than the control unit.

### CROSS REFERENCE TO RELATED APPLICATION

This application is based on, and claims the benefit of priority from Japanese Patent Application No. 2024-171794 on September 30, 2024. The entire disclosure of the above application is incorporated herein by reference.

### TECHNICAL FIELD

The present disclosure relates to a fundus imaging apparatus for capturing an image of the fundus of a subject eye.

### BACKGROUND ART

As a fundus imaging apparatus for capturing images of the fundus of a subject eye, composite devices capable of capturing both a color image as a front image of the fundus and an OCT image have been known (e.g., JP 2023-049396 A). Generally, such a composite device is configured as a separated type device, comprising a main unit that includes an imaging unit (also referred to as a head) incorporating various optical systems, and an external control device connected to the main unit. The external control device functions as a control unit for imaging, display, analysis processing, and the like.

### SUMMARY

Such a composite device as described above has recently been introduced not only in ophthalmology clinics but also in a wide range of facilities such as health screening centers and optical shops.

To make the device easier to install in various facilities, it is conceivable to integrate the control unit into the main device body—similar to other ophthalmic examination devices such as autorefractors—while further reducing the installation footprint. However, when considering placement of the control unit inside the imaging unit that houses various optical systems to reduce an installation area, the issue of heat generated by the control unit affecting the optical systems became apparent. Specifically, such composite devices require a certain amount of space within the imaging unit to accommodate the optical systems. Moreover, since the control unit handles significantly larger data volumes for imaging control, display control, and analysis processing compared to other ophthalmic devices, if the optical systems and the control unit are placed within the same imaging unit, it is necessary to sufficiently reduce the impact on the optical systems caused by the heat generated from the control unit.

The present disclosure has been made in view of the above, and provides a fundus imaging apparatus that enables space-saving installation while suppressing thermal changes in optical systems.

In a first aspect of the present disclosure, a fundus imaging apparatus includes: a base; a display unit; an imaging optical system configured to capture at least a color image as a fundus front image of a subject eye; an OCT optical system configured to acquire OCT data of a fundus of the subject eye; a control unit configured to execute at least (i) imaging control of the imaging optical system and the OCT optical system, (ii) display control processing of the color image and an OCT image based on the OCT data, and (iii) analysis processing of the OCT data; and an imaging unit housing the imaging optical system, the OCT optical system, and the control unit therein, the imaging unit being disposed on the base. The control unit is disposed above the imaging optical system and the OCT optical system in the imaging unit. The imaging unit includes an exhaust port that discharges air heated by the control unit. The exhaust port is located at the same height as the control unit or at a position higher than the control unit.

In a second aspect of the present disclosure, a fundus imaging apparatus includes: a base; a display unit; an imaging optical system configured to capture at least a color image as a fundus front image of a subject eye; an OCT optical system configured to acquire OCT data of a fundus of the subject eye; a control unit configured to execute at least (i) imaging control of the imaging optical system and the OCT optical system, (ii) display control processing of the color image and an OCT image based on the OCT data, and (iii) analysis processing of the OCT data; and an imaging unit housing the imaging optical system, the OCT optical system, and the control unit therein, the imaging unit being disposed on the base. The control unit is disposed above the imaging optical system and the OCT optical system in the imaging unit.

### BRIEF DESCRIPTION OF DRAWINGS

FIG. 1 is a right side view of a fundus imaging apparatus.

FIG. 2 is a left side view of the fundus imaging apparatus.

FIG. 3 is a schematic diagram of various optical systems.

FIG. 4 is a configuration diagram of an imaging optical system.

FIG. 5 is a configuration diagram of a control unit.

FIG. 6 is a front view of the fundus imaging apparatus.

### DETAILED DESCRIPTION OF THE PREFERRED EMBODIMENTS

### Overview

One embodiment of the fundus imaging apparatus according to the present disclosure will be described. Each embodiment may be applied independently or in combination with all or part of other embodiments. For example, the items classified using brackets <> below may be used independently or in combination.

The fundus imaging apparatus according to the present embodiment includes a so-called fundus camera. Using an imaging optical system described later, the fundus imaging apparatus captures a front image of the fundus in color and displays the image on a display unit. Furthermore, the fundus imaging apparatus includes an optical coherence tomography (OCT) device. The fundus imaging apparatus also functions as an information analysis unit that analyzes OCT data acquired from tissues (e.g., the fundus) captured using the OCT optical system described later, and generates OCT images (e.g., tomographic images, motion contrast images, etc.) as the analysis results of the OCT data.

The fundus imaging apparatus in this embodiment includes an imaging optical system (e.g., an imaging optical system 200). All or part of the imaging optical system may be housed in the imaging unit described later. The fundus imaging apparatus also includes an OCT optical system (e.g., an OCT optical system 201), which may also be entirely or partially housed in the imaging unit.

### Imaging Optical System

The imaging optical system is an optical system configured to capture at least a color image as a front image of the fundus of the subject eye. The system includes an illumination optical system for irradiating the fundus of the subject eye with visible light, and a light-receiving optical system including a light-receiving element for receiving reflected light of the illumination light from the fundus. For example, illumination light may be guided to the fundus via an objective optical system. The imaging optical system forms both an irradiation region and a light-receiving region for imaging light at the pupil of the subject eye. A front image of the fundus of the subject eye is captured by transmitting and receiving the imaging light through these regions. The imaging optical system may also serve as a fundus observation optical system that acquires observation images of the fundus.

### OCT Optical System

The OCT optical system is an optical system configured to acquire OCT data of the fundus using interference between measurement light and reference light irradiated onto the subject eye. The OCT optical system housed in the imaging unit may include at least a measurement optical system that guides measurement light—provided from a beam splitter—to the fundus of the subject eye. For example, the measurement light may be guided to the subject eye through an objective optical system. Additionally, the OCT optical system housed in the imaging unit may include a detector for detecting interference light resulting from the reflection light of the measurement light and the reference light.

The fundus imaging apparatus may adopt a time-domain OCT (TD-OCT) configuration. Alternatively, the fundus imaging apparatus may adopt a Fourier-domain OCT configuration, such as a spectral-domain OCT (SD-OCT) or a swept-source OCT (SS-OCT). In SD-OCT, a low-coherence light source (a broadband light source) is used as a measurement light source, and a spectroscopic optical system (a spectrometer) is provided near the light-receiving element in a path of interference light to spectrally separate the interference light into its frequency (wavelength) components. In SS-OCT, a wavelength scanning light source (a wavelength-tunable light source) that rapidly changes an emission wavelength over time is used as the measurement light source.

A technology of the present embodiment may be applied to at least one of: an intensity OCT that detects a reflection intensity of a subject eye; an OCT angiography that detects motion contrast data of the subject eye (e.g., Doppler OCT); a polarization-sensitive OCT (PS-OCT); or a multifunction OCT that combines both intensity OCT and PS-OCT.

In the imaging optical system and the OCT optical system described above, at least some components may be shared. For example, a single objective optical system that is commonly used to (i) guide illumination light of the imaging optical system to the subject eye and to (ii) guide measurement light of the OCT optical system to the subject eye may be provided. In such a case, an optical path coupling member (e.g., a beam splitter 221) for combining optical paths of the imaging optical system and the OCT optical system may be provided, and the single objective optical system may be disposed between a subject eye and the optical path coupling member. The optical path coupling member may be, for example, a beam splitter, a half mirror, or a dichroic mirror.

### Control Unit

A control unit (e.g., a control unit 100) executes imaging control of the imaging optical system and the OCT optical system. The control unit also performs display control processing of a color image captured by the imaging optical system and an OCT image based on OCT data acquired by the OCT optical system. Furthermore, the control unit performs analysis processing of the OCT data. The analysis may include at least one of: a layer detection process (a segmentation process) of an OCT image, a thickness analysis process of tissue, or a density analysis process of tissue. The control unit may also perform other types of control different from the imaging, display, and analysis processing.

The fundus imaging apparatus of the present embodiment includes a display unit (e.g., a display unit 125), a base (e.g., a base 7), and an imaging unit (e.g., an imaging unit 3) disposed on the base. The display unit may be provided on a main body of the fundus imaging apparatus or may be connected to the main body via a wired or wireless connection. The imaging unit internally houses an imaging optical system, an OCT optical system, and a control unit. Within the imaging unit, the imaging optical system and the OCT optical system may each be protected by either individual enclosures or a common enclosure that collectively covers both systems. By placing the control unit within the imaging unit, an external control device becomes unnecessary, allowing the fundus imaging apparatus to operate in standalone mode for imaging, display, analysis, and other functions, thereby achieving a compact design. The fundus imaging apparatus may further include a drive unit (e.g., a drive unit 8) that moves the imaging unit relative to the base.

In the fundus imaging apparatus, the control unit is disposed above the imaging optical system and the OCT optical system. For example, if the imaging optical system and the OCT optical system are arranged in parallel, the control unit may be placed above either of them. Alternatively, if one optical system is stacked on top of the other, the control unit may be disposed above the upper one of the optical systems. A term "above" refers to spatial positioning, such as above at least part of an installation surface of the imaging optical system and the OCT optical system. Further, the term “above” may be “above” within the installation surface of the imaging optical system and the OCT optical system. However, placing the control unit above these optical systems—beyond an area of their installation surfaces—may increase a width of the imaging unit. Therefore, from a space-saving standpoint of the imaging unit, it is preferable to position the control unit above the optical systems within their respective installation areas.

Even when the control unit and the optical systems are placed within the imaging unit, positioning the control unit above the optical systems helps suppress thermal changes in the optical systems. Processing tasks such as imaging, display, and analysis handled by the control unit are computationally intensive. If the control unit heats up to high temperatures (e.g., 50 to 85°C), the heat may cause thermal expansion of components that hold parts of the optical systems. However, since air heated by the control unit has difficulty reaching the optical systems, displacement of an optical axis and changes in an optical path length due to thermal effects can be minimized.

In the fundus imaging apparatus, the control unit may be positioned between a subject eye and an optical path coupling member, and also positioned higher than an optical axis of an objective optical system that guides light to a fundus. In this case, rising hot air generated by the control unit can be directed away from the objective optical system, thereby reducing a likelihood of thermal effects on the objective optical system.

In the fundus imaging apparatus, the control unit may be disposed above an enclosure that covers the imaging optical system and the OCT optical system. This configuration places the control unit above all components constituting the imaging optical system and the OCT optical system, further reducing a likelihood that either optical system will be affected by thermal changes.

The fundus imaging apparatus of the present embodiment may include a detector of the OCT optical system housed within the imaging unit. As one example, in the case of SD-OCT, a spectrometer may be used as the detector. The spectrometer comprises a plurality of optical elements and a mount portion that hods the plurality of optical elements. Each of the plurality of optical elements includes a grating that disperses interference light between measurement light and reference light, and a light-receiving element that detects the dispersed interference light. For example, if a detector is housed in a base, wiring between a control unit and the detector must account for movement of the imaging unit, which tends to complicate the configuration. In contrast, when a detector is disposed not in the base but in the imaging unit together with the control unit, a wiring configuration for electrically connecting the detector and the control unit tends to be simplified. Furthermore, when a display unit is attached to the imaging unit, a wiring configuration for electrically connecting the control unit and the display unit also tends to be simplified.

Additionally, when the detector of the OCT optical system is disposed not in the base but in the imaging unit, the base can be made more compact, and an installation footprint of the fundus imaging apparatus can be reduced. Since a position of the detector within the imaging unit is below the control unit that serves as a heat source, even if the detector is disposed with the control unit in the imaging unit, heat from the control unit is unlikely to affect the detector.

The fundus imaging apparatus of the present embodiment includes, in the imaging unit, an exhaust port (e.g., an exhaust port 5) for discharging air. For example, the exhaust port may be located at an upper portion of the imaging unit. Alternatively, for example, the exhaust port may be located at a position at the same height as the control unit, or at a position higher than the control unit.

The fundus imaging apparatus of the present embodiment includes, within the imaging unit, an air-blowing mechanism (e.g., a fan 6) provided on a side surface of the control unit to generate an airflow. Air flows within the imaging unit due to the air-blowing mechanism and is discharged from the imaging unit through the exhaust port. As the air-blowing mechanism, a fan, a blower, or the like may be used. This allows air heated by the control unit to be efficiently discharged.

The air-blowing mechanism may be one or more air-blowing mechanisms. For example, the air-blowing mechanism may be a plurality of air-blowing mechanisms arranged in parallel along a side surface of the control unit. As one example, the air-blowing mechanisms may be arranged in parallel along a longitudinal direction of the control unit on its side surface. In such a configuration, since the air-blowing mechanisms are aligned with the control unit, all blown air can be directed toward the control unit, resulting in improved cooling efficiency.

When an imaging unit includes the exhaust port and the air-blowing mechanism is disposed on a side surface of the control unit, the exhaust port may be provided at the same height as the control unit. In this case, the exhaust port is positioned at the same height as the air-blowing mechanism. As a result, recirculation of hot air within the imaging unit can be suppressed, and thermal changes in optical systems can be further reduced.

### Embodiment

An embodiment of the fundus imaging apparatus according to the present embodiment will be described. The fundus imaging apparatus 1 captures a color front image of a fundus Er of a subject eye E. The fundus imaging apparatus 1 also captures an OCT image of the fundus Er of the subject eye E. The OCT image may be a tomographic image, a front image (an EnFace image), a motion contrast image, or the like.

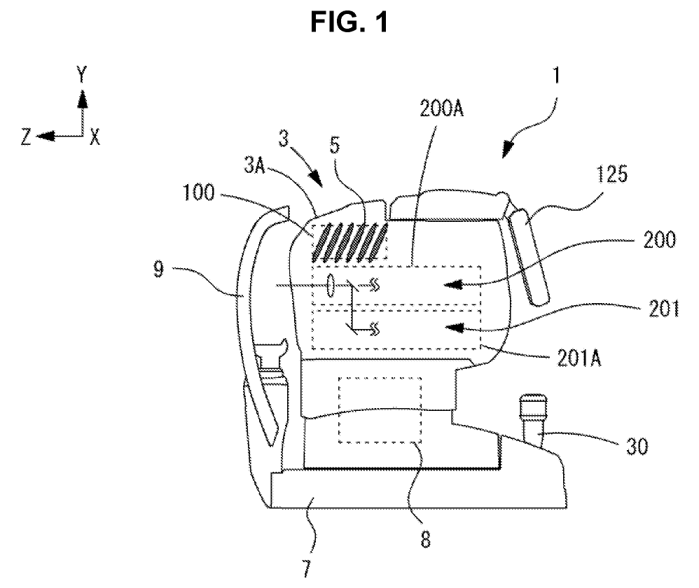

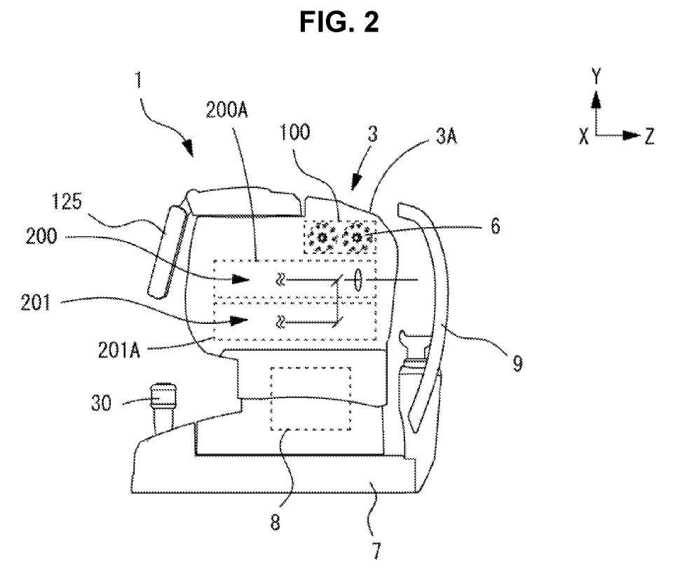

FIG. 1 and FIG. 2 are external views of the fundus imaging apparatus 1. FIG. 1 is a right side view of the fundus imaging apparatus 1. FIG. 2 is a left side view of the fundus imaging apparatus 1. In this embodiment, a side of the fundus imaging apparatus 1 where a face support portion 9 is disposed is referred to as the front, and the opposite side is referred to as the rear. With respect to the front of the fundus imaging apparatus 1, a left-right direction is referred to as an X direction, an up-down direction as a Y direction, and a front-rear direction as a Z direction.

The fundus imaging apparatus 1 includes a base 7, an imaging unit 3, a drive unit 8, the face support portion 9, an exhaust port 5, an operation unit 30, a display unit 125, a control unit 100, and the like. The base 7 supports the imaging unit 3. The base 7 also supports the face support portion 9. The imaging unit 3 serves as an examination unit. The imaging unit 3 is covered by a cover 3A. The drive unit 8 moves the imaging unit 3 in X, Y, and Z directions relative to the base 7. The face support portion 9 fixes a subject’s face in place. The exhaust port 5 is provided on the cover 3A of the imaging unit 3. The exhaust port 5 allows air flowing within the imaging unit 3 (inside the cover 3A) to be discharged to outside the imaging unit 3 (outside the cover 3A), which will be described later.

The operation unit 30 outputs an operation signal corresponding to an operation instruction input via the operation unit 30 to the control unit 10. For example, as the operation unit 30, at least one of a mouse, a joystick, a keyboard, a touch panel, or the like may be used.

The display unit 125 is a display disposed in the imaging unit 3. The display unit 125 may also function as the operation unit 30. For example, the display unit 125 may include a touch panel function and thereby serve concurrently as the operation unit 30.

The imaging unit 3 (the cover 3A) houses various optical systems (for example, an imaging optical system 200 and an OCT optical system 201) and the control unit 100. The imaging optical system 200 is protected by an enclosure 200A that covers the imaging optical system 200. The OCT optical system 201 is protected by an enclosure 201A that covers the OCT optical system 201. It is also possible to house and protect both the imaging optical system 200 and the OCT optical system 201 by a single enclosure. The imaging unit 3 may further house a driver for controlling a light scanner 108 (described later) included in the OCT optical system 201.

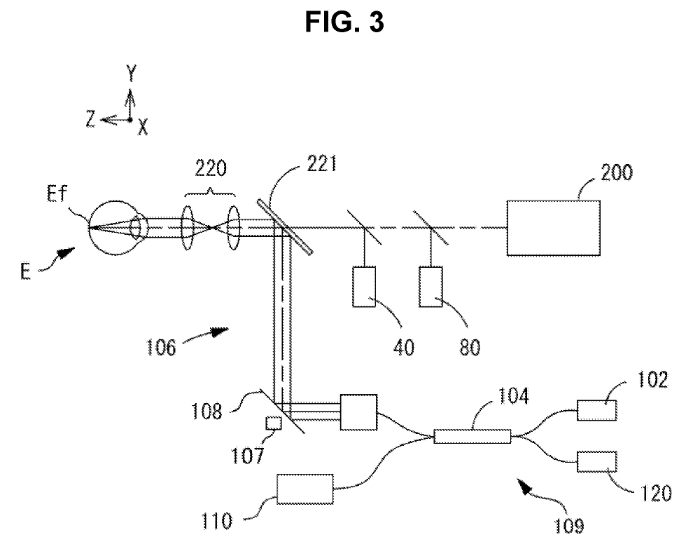

FIG. 3 is a schematic diagram of various optical systems. As the various optical systems, the imaging optical system 200, the OCT optical system 201, an index projection optical system 70, an anterior ocular observation optical system 40, a fixation target projection optical system 80, and the like are provided. In this embodiment, at least the imaging optical system 200 and the OCT optical system 201 share the single objective optical system 220. Further, optical paths of the imaging optical system 200 and the OCT optical system 201 are combined and made coaxial by a beam splitter 221, which is one example of the optical path coupling member.

### Imaging Optical System

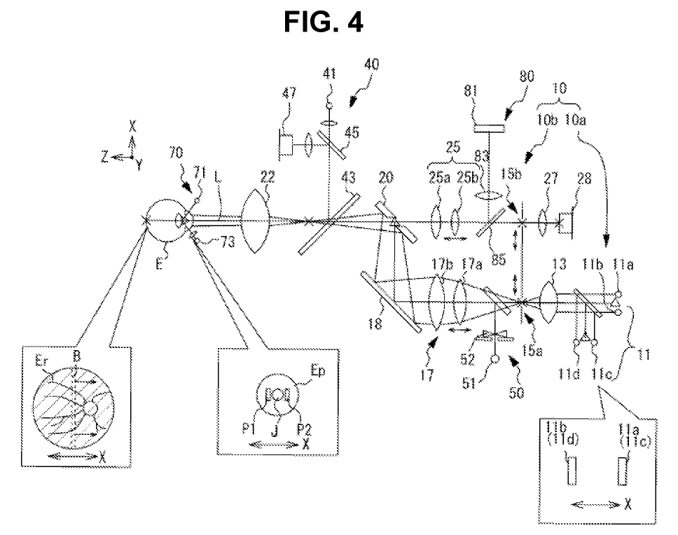

FIG. 4 is a configuration diagram of the imaging optical system 200. In FIG. 4, the objective optical system 220 is schematically represented by a single objective lens 22 for convenience. In FIG. 4, a pupil conjugate position that is conjugate with a pupil of a subject eye E is marked with “Δ” on the optical axis, and a fundus conjugate position is marked with “x” on the optical axis.

The imaging optical system 200 includes an illumination optical system 10a and a light-receiving optical system 10b. The illumination optical system 10a includes a light source unit 11, a lens 13, a slit-shaped member 15a, lenses 17a and 17b, a mirror 18, a perforated mirror 20, and the objective lens 22. The light-receiving optical system 10b includes the objective lens 22, a perforated mirror 20, lenses 25a and 25b, a slit-shaped member 15b, an image sensor 28, and the like.

The light source unit 11 includes a plurality of types of light sources having different wavelength bands. For example, the light source unit 11 includes visible light sources 11a and 11b, and infrared light sources 11c and 11d. Thus, in the light source unit 11 of the present embodiment, two light sources are provided for each wavelength. The two light sources of the same wavelength are disposed away from an optical axis L on a pupil conjugate plane. The two light sources are arranged along the X direction, which is a scanning direction in FIG. 4, and are arranged symmetrically with respect to the optical axis L. An outer shape of the two light sources may be a rectangular shape longer in a direction intersecting the scanning direction than in the scanning direction.

Light from the two light sources passes through the lens 13 and is irradiated onto the slit-shaped member 15a. In the present embodiment, the slit-shaped member 15a includes a light-transmitting portion (opening) that is elongated along a Y direction. As a result, on a fundus conjugate plane, illumination light is formed in a slit shape. A region illuminated in a slit shape on a fundus Er is indicated by reference sign B in the drawings.

The slit-shaped member 15a is displaced by a drive unit (not shown) so that the light-transmitting portion traverses the optical axis L in the X direction. This enables scanning of the illumination light in the present embodiment. In the present embodiment, scanning is also performed on the light-receiving side by the slit-shaped member 15b. In this embodiment, the slit-shaped members on the projection side and the light-receiving side are driven in coordination by the single drive unit (an actuator). Thus, a scanning unit including the slit-shaped members 15a and 15b is formed. The scanning unit may be, for example, an optical chopper. For details of an optical system adopting an optical chopper, reference may be made to JP 2019-118721 A filed by the present applicant, which will be incorporated herein by reference.

In the illumination optical system 10a, images of each light source are relayed by the optical systems from the lens 13 to the objective lens 22 and are formed on a pupil conjugate plane. That is, on the pupil conjugate plane, pupil images of the two light sources are formed at positions separated in a scanning direction. Thus, in this embodiment, two illumination regions P1 and P2 on the pupil conjugate plane are formed as images of the two light sources.

Also, slit-shaped light that has passed through the slit-shaped member 15a is relayed by the optical system from the lens 17a to the objective lens 22 and is focused on the fundus Er. As a result, illumination light is formed in a slit shape on the fundus Er. The illumination light is reflected at the fundus Er and exits through a pupil Ep.

The perforated mirror 20 serves as the optical path coupling member that combines optical paths of the illumination optical system 10a and the light-receiving optical system 10b. The perforated mirror 20 reflects illumination light from the light source unit 11 toward the subject eye E, and allows a portion of fundus-reflected light from the subject eye E that passes through an opening to pass toward the image sensor 28. Various beam splitters other than the perforated mirror 20 may be used.

An opening of the perforated mirror 20 is conjugate with a pupil of a subject eye. Therefore, fundus-reflected light used for imaging is limited to only a portion that passes through an image of the opening (the pupil image) on the pupil of the subject eye. Thus, an image of the opening on the pupil of the subject eye becomes a light-receiving region J in this embodiment. The light-receiving region J is formed between the two illumination regions P1 and P2 (images of the two light sources). Also, as a result of appropriately setting imaging magnification of each image, a diameter of the opening, and a distance between the two light sources, the light-receiving region J and the two illumination regions P1 and P2 are formed on the pupil such that they do not overlap with each other.

Fundus-reflected light that has passed through the objective lens 22 and the opening of the perforated mirror 20 is focused onto a fundus conjugate plane via lenses 25a and 25b, thereby forming an image of a slit-shaped region of a fundus Er. At this time, since a light-transmitting portion of the slit-shaped member 15b is disposed at the image formation position, unnecessary light is removed.

The image sensor 28 is disposed at the fundus conjugate plane. In this embodiment, a relay optical system 27 is provided between the slit-shaped member 15b and the image sensor 28. As a result, both the slit-shaped member 15b and the image sensor 28 are in a conjugate relationship with the fundus. Accordingly, both removal of unnecessary light and image formation are performed effectively. Alternatively, the relay optical system 27 between the image sensor 28 and the slit-shaped member 15b may be omitted, and the two components may be arranged in close proximity. In this embodiment, a device with a two-dimensional light-receiving surface is used as the image sensor 28. For example, a CMOS or a two-dimensional CCD may be used. An image of a slit-shaped region of the fundus Er, which has been formed via the light-transmitting portion of the slit-shaped member 15b, is projected onto the image sensor 28. The image sensor 28 is sensitive to both infrared light and visible light.

In the present embodiment, as slit-shaped illumination light is scanned over a fundus Er, an image (slit-shaped image) of the scanning position on the fundus Er is sequentially projected line by line onto the image sensor 28. In this way, a full image of a scanning range is time-divisionally projected onto the image sensor 28. As a result, a front image (a two-dimensional reflection image) of the fundus is captured as a complete image of the scanning range.

In the present embodiment, a scanning unit in the light-receiving optical system 10b is a mechanical device that scans a slit, but it is not limited to this. For example, the scanning unit on a side of the light-receiving optical system 10b may be a device that scans the slit electronically. As one example, when the image sensor 28 is a CMOS sensor, scanning of the slit may be realized by a rolling shutter function of the CMOS. In this case, by displacing a region exposed on an imaging surface in synchronization with a scanning unit in the illumination optical system 10a, unnecessary light can be removed while efficiently capturing an image. Additionally, a liquid crystal shutter or the like may be used as a scanning unit that electronically scans the slit.

The imaging optical system 200 includes a diopter correction unit. In the present embodiment, the diopter correction unit (diopter correction optical systems 17 and 25) is provided in each of an independent optical path of the illumination optical system 10a and an independent optical path of the light-receiving optical system 10b. However, the diopter correction unit may be provided in a common optical path shared by the illumination optical system 10a and the light-receiving optical system 10b.

The imaging optical system 200 may further include an index projection optical system 50. The index projection optical system 50 projects two split indexes onto a fundus Er as focus indexes. The split indexes are used for focus detection.

### OCT Optical System

The OCT optical system 201 (see FIG. 3) includes a measurement light source 102, a coupler (optical splitter) 104, a measurement optical system 106, a scanning unit (light scanner) 108, a reference optical system 110, a light-receiving element (detector) 120, and the like. For example, the measurement light source 102, the measurement optical system 106, the reference optical system 110, and the light-receiving element 120 are connected to the coupler 104 via optical fibers.

The OCT optical system 201 splits light emitted from the measurement light source 102 into measurement light (sample light) and reference light by the coupler 104. The OCT optical system 201 guides the measurement light to a tissue (here, a fundus Ef) of the subject eye E via the measurement optical system 106, and guides the reference light to the reference optical system 110. The OCT optical system 201 causes the light-receiving element 120 to receive interference light formed by combining measurement light reflected by the tissue and the reference light.

The measurement light source 102 emits low-coherence light used as measurement light and reference light. Light emitted from the measurement light source 102 is split into measurement light and reference light by the coupler 104. The measurement light passes through an optical fiber and is emitted into the air. The measurement light emitted into the air is focused onto the tissue via the measurement optical system 106, including the light scanner 108. Measurement light reflected by the tissue returns to the optical fiber along the same optical path.

The light scanner 108 scans the measurement light in two-dimensional directions (X and Y directions) on the tissue. For example, the light scanner 108 is disposed at a position substantially conjugate with a pupil of the subject eye E. As one example, the light scanner 108 includes two galvanometer mirrors. Reflection angles of the galvanometer mirrors are arbitrarily adjusted by the drive mechanism 107. As a result, a reflection direction of the measurement light changes, and the measurement light is irradiated onto an arbitrary position on the tissue. In other words, the irradiation position of the measurement light on the tissue is changed by the light scanner 108. It goes without saying that a configuration of the light scanner 108 may be modified. For example, a polygon mirror, a resonant scanner, or an acousto-optic modulator (AOM) may be used as the light scanner 108.

The reference optical system 110 generates reference light to be combined with measurement light reflected by a tissue. The reference optical system 110 may be of the Michelson type or the Mach-Zehnder type. The reference optical system 110 reflects light incident from the coupler 104 using a reflective optical system (for example, a reference mirror), returns it to the coupler 104, and guides it to the light-receiving element 120. As another example, the reference optical system 110 may transmit the light incident from the coupler 104 without reflection and guide it to the light-receiving element 120.

The reference optical system 110 can change an optical path length difference between measurement light and reference light by moving an optical member in the optical path. In the present embodiment, the optical path length difference is changed by moving a reference mirror in an optical axis direction. A configuration for changing the optical path length difference may also be provided in an optical path of the measurement optical system 106.

The light-receiving element 120 detects an interference signal resulting from the combination of measurement light and reference light. For example, the light-receiving element 120 includes a spectroscopic optical system (a spectrometer) that disperses the interference signal into each wavelength component (each frequency component). The spectrometer, for example, includes a diffraction grating and a line sensor. The interference signal detected by the light-receiving element 120 is output to the control unit 10.

In a Fourier domain OCT, for example, spectral intensity (a spectral interference signal) of interference light is detected by the light-receiving element 120. A complex OCT signal is obtained by performing Fourier transform on the spectral intensity data. An absolute value of amplitude in the complex OCT signal is calculated to obtain a depth profile (A-scan signal) within a predetermined range. A B-scan signal is obtained by arranging depth profiles at each measurement point, which were scanned by the measurement light using the light scanner 108. OCT image data (tomographic image data) is obtained based on the B-scan signal. Also, three-dimensional OCT image data (three-dimensional tomographic image data) may be obtained by scanning the measurement light in two-dimensional directions on a tissue and arranging the B-scan signals of each scan line. Furthermore, Enface OCT image data, which is a view of the tissue from a front direction along an optical axis of the measurement light, may also be obtained from the three-dimensional OCT image data.

Moreover, motion contrast data may be obtained from two or more OCT signals acquired at different timings (different times) from the same region of the tissue. That is, motion contrast data is obtained by performing analysis processing on multiple complex OCT signals. For example, when measurement light is scanned along one scan line and motion contrast data at each scan position is arranged, two-dimensional motion contrast data is obtained. Also, by scanning the measurement light in two-dimensional directions (i.e., XY directions along the optical axis of the measurement light), three-dimensional motion contrast data may be obtained. Furthermore, Enface motion contrast data of the tissue, as viewed from the front, may be obtained from the three-dimensional motion contrast data.

### Index Projection Optical System

The index projection optical system 70 projects alignment indices onto a cornea of the subject eye E. The index projection optical system 70 includes a first index projection optical system 73 and a second index projection optical system 71. The first index projection optical system 73 projects collimated light (infinite distance light). The second index projection optical system 71 projects divergent light (finite distance light). The first index projection optical system 73 is symmetrically arranged at 0 and 180 degrees relative to the optical axis L. The second index projection optical system 71 is arranged concentrically at 45, 90, 135, 225, 270, and 315 degrees around the optical axis L.

### Anterior Ocular Observation Optical System

The anterior ocular observation optical system 40 captures an anterior ocular segment of a subject eye E and acquires an anterior ocular observation image. The anterior ocular observation optical system 40 illuminates the anterior ocular segment with infrared light and captures a front image of the anterior ocular segment. The anterior ocular observation optical system includes a light source 41, a half mirror 45, an imaging element 47, a dichroic mirror 43, the objective lens 22, and the like. For example, the light source 41 is an infrared light source that illuminates the subject eye E. For example, the imaging element 47 is a two-dimensional imaging element and is disposed at a position optically conjugate with a pupil Ep. The dichroic mirror 43 and the objective lens 22 are shared with the imaging optical system 200.

### Fixation Target Projection Optical System

The fixation target projection optical system 80 projects (presents) a fixation target to the subject eye E. When the fixation target is perceived by a subject, movement of a line of sight is suppressed. The index projection optical system 80 may include, for example, a fixation target unit 81 and a relay lens 83, and may share an optical path of the imaging optical system 200 from a dichroic mirror 85 to the objective lens 22. The fixation target unit 81 emits visible light.

### Control Unit

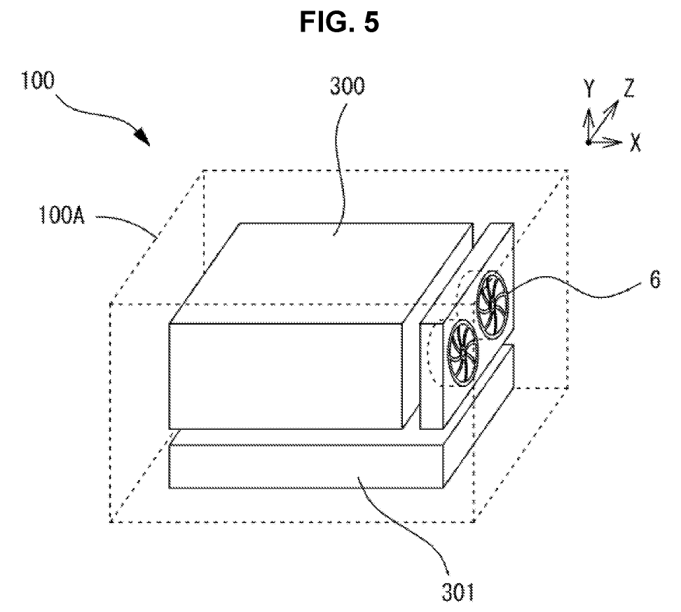

FIG. 5 shows a configuration diagram of the control unit 100. The control unit 100 has functions necessary for controlling each part and performing various arithmetic and analysis processes. The control unit 100 is protected by the enclosure 100A that covers the control unit 100. The control unit 100 includes at least a single-board computer (SBC) 301. The SBC 301 includes a CPU, a RAM, a ROM, a storage unit, and the like. The CPU controls the fundus imaging apparatus 1. The CPU may be configured with one or a plurality of processors. Various information is temporarily stored in the RAM. Various programs, initial values, and the like are stored in the ROM. The storage unit is a non-transitory storage medium capable of retaining stored contents even when power supply is interrupted. For example, a hard disk drive, a flash ROM, a USB memory, or the like may be used as the storage unit.

Furthermore, the control unit 100 includes a heat sink 300, fans 6, and the like. The heat sink 300 is disposed above the SBC 301. The heat sink 300 absorbs heat generated by the SBC 301 and dissipates it into the air. The fan 6 generates an air flow by blowing air. The fans 6 are arranged in parallel on a side surface of the control unit 100 (that is, a side surface of the enclosure 100A). In the present embodiment, two fans 6 are arranged in parallel in a longitudinal direction (Z direction in FIG. 5) on the side surface of the control unit 100 (heat sink 300).

The control unit 100 is electrically connected to the drive unit 8, the operation unit 30, the display unit 125, the imaging optical system 200, the OCT optical system 201, the index projection optical system 70, the anterior ocular observation optical system 40, the fixation target projection optical system 80, the storage unit, and the like.

### Arrangement of Optical Systems and Control Unit, and Air Flow Path

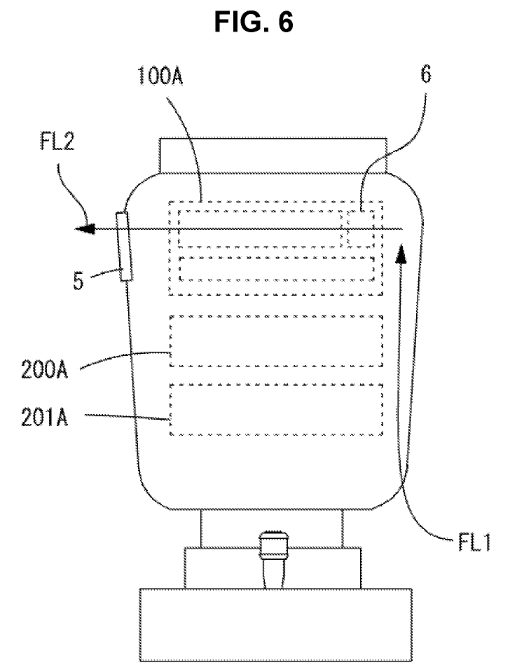

FIG. 6 is a front view of the fundus imaging apparatus 1. With reference to FIG. 6, arrangement of the optical systems and the control unit 100, and an air flow path will be described. Note that in FIG. 6, illustration of the display unit 125 is omitted.

In the imaging unit 3 (cover 3A), the imaging optical system 200 (enclosure 200A) is disposed above the OCT optical system 201 (enclosure 201A). Also, in the imaging unit 3, the control unit 100 (enclosure 100A) is disposed above the imaging optical system 200 (enclosure 200A). That is, the control unit 100 is positioned above the installation surface of the enclosure 201A and above the installation surface of the enclosure 200A. In addition, the control unit 100 is disposed above the optical axis of the objective optical system 220 in the OCT optical system 201 and the imaging optical system 200. Furthermore, the control unit 100 is disposed at substantially the same height as the exhaust port 5. In other words, the exhaust port 5 is overlapped with the control unit 100 in X direction.

The imaging unit 3 (the cover 3A) is not sealed, and a gap (not shown) is provided in a lower portion of the imaging unit 3. Outside air (air) flows into the imaging unit 3 through this gap serving as an intake port, and is discharged to the outside of the imaging unit 3 via the fans 6, the heat sink 300, and the exhaust port 5. More specifically, the outside air flows into the imaging unit 3, flows along an air flow path FL1, and reaches the fans 6 of the control unit 100. In the control unit 100, the SBC 301 generates heat associated with control of the fundus imaging apparatus 1, and the heat is conducted to the heat sink 300. The heat sink 300 dissipates (radiates) this heat from its surface to the surroundings. Accordingly, heated air remains in the upper portion of the imaging unit 3, that is, around the control unit 100. After flowing along the air flow path FL1, the outside air is blown by the fans 6, and together with air heated by the SBC 301, passes along the air flow path FL2 through an upper portion of the imaging optical system 200 (the enclosure 200A). Furthermore, the outside air is discharged to the outside of the imaging unit 3 from the exhaust port 5 by an exhaust action (exhaustion force) due to the blowing of the fans 6.

According to the present embodiment, since the control unit 100 is disposed above the OCT optical system 201 and the imaging optical system 200, the control unit 100 is less susceptible to thermal changes, such as optical axis deviation, caused by rising heated air from the control unit 100, and a certain level of accuracy can be maintained. Also, since the control unit 100 is disposed at substantially the same height as the exhaust port 5, heated air from the control unit 100 does not circulate within the imaging unit 3. Therefore, heat does not spread to each optical system, and the control unit 100 can be cooled efficiently.

### Operation

Operation control of the fundus imaging apparatus 1 will be described.

An examiner newly registers or calls identification information (ID, name, age, etc.) of a subject, and sets a shooting mode to be applied to acquisition of a fundus front image or a tomographic image by OCT of the subject eye. The examiner also operates the operation unit 30 and presses a start switch to begin imaging of the subject eye. The control unit 100 controls various optical systems based on an operation instruction from the operation unit 30.

For example, the control unit 100 controls the fixation target projection optical system 80 to turn on a fixation lamp included in the fixation target unit 81. As a result, a line of sight of the subject eye (in other words, a fixation position) is guided onto the optical axis L. The control unit 100 also controls the index projection optical system 70 to project the alignment indices onto a cornea. Furthermore, the control unit 100 controls the anterior ocular observation optical system 40 to sequentially acquire anterior ocular observation images. For example, such anterior ocular observation images may be displayed on the display unit 125.

In the present embodiment, based on an anterior ocular observation image of the subject eye E, a position of the imaging unit 3 relative to the subject eye E is automatically moved. The control unit 100 detects an alignment index image included in the anterior ocular observation image by performing image processing on the anterior ocular observation image. The control unit 100 also controls the drive unit 8 to adjust an alignment state of the imaging unit 3 relative to the subject eye E based on the alignment index image.

The examiner, when the subject eye E is fixating on the fixation lamp and automatic alignment has been completed, presses a Capture button. The control unit 100, based on an operation signal input from the Capture button, controls the imaging optical system 200 to perform focus adjustment and capture a fundus front image.

In addition, based on an operation signal input from the Capture button, the control unit 100 controls the OCT optical system 201 to perform optimization processing, scan the fundus Er with measurement light, and acquire an OCT signal (interference signal).

Subsequently, the control unit 100 analyzes the OCT signal and acquires OCT data based on the OCT signal. Furthermore, the control unit 100 obtains an OCT image, which is an image created from the OCT data. The control unit 100 also acquires segmentation results in which layers and boundaries of tissue are segmented based on the OCT data. Moreover, the control unit 100 may generate an analysis map using the segmentation results (as one example, a thickness map showing a distribution of layer thicknesses of the fundus in two dimensions). The display unit 125 displays these OCT images, the segmentation results, and the analysis map.

The more fundus Er images are captured by the fundus imaging apparatus 1, and the longer the apparatus is continuously used, the higher the load on the control unit 100 becomes, making the control unit 100 more likely to generate heat. However, within the imaging unit 3, air heated by the control unit 100 is, together with air entering from outside the imaging unit 3, appropriately discharged from the exhaust port 5 through the above-described air flow path without striking various optical systems.

As described above, in the fundus imaging apparatus according to the present embodiment, since the control unit is disposed not in the base but in the imaging unit, the base can be downsized, and the installation area of the apparatus can be reduced. Conventionally, a combined apparatus for capturing both a fundus front image by a color image and an OCT tomographic image required an external control device (typically a PC) that executes at least one of imaging control, display processing, and analysis processing. In contrast, in the present embodiment, the control unit that executes at least imaging control of the imaging optical system and the OCT optical system, display control processing of an OCT tomographic image based on the color image and OCT data, and analysis processing of the OCT data is disposed in the imaging unit. Accordingly, the apparatus can be realized compactly as a standalone device that performs imaging, display, and analysis without requiring an external PC.

However, in this case, since the load of processing performed by the control unit (imaging control of the imaging optical system and the OCT optical system, display control processing of the OCT tomographic image based on the color image and OCT data, and analysis processing of the OCT data) is high, the control unit generates heat at a high temperature (for example, approximately 50 to 85°C). In view of this, within the imaging unit, since the control unit is disposed above the imaging optical system and the OCT optical system, air heated by the control unit hardly reaches the imaging optical system and the OCT optical system. Therefore, even though the control unit is disposed together with the imaging optical system and the OCT optical system in the imaging unit, thermal changes in the imaging optical system and the OCT optical system are less likely to occur. As typical thermal changes, expansion of parts holding each portion of the optical system may occur. Due to such expansion, optical axis deviation, changes in a total length of the optical path, and the like may occur.

In the fundus imaging apparatus of the present embodiment, since the control unit is disposed above the optical axis of the objective optical system, heated air generated by the control unit rises, and at least the objective optical system is less likely to be affected by thermal changes.

In the fundus imaging apparatus of the present embodiment, since the control unit is disposed above the enclosures covering the imaging optical system and the OCT optical system, the control unit is placed above all components constituting the optical systems. Accordingly, each optical system is less susceptible to thermal changes.

In the fundus imaging apparatus of the present embodiment, the detector of the OCT optical system is disposed in the imaging unit. When the detector is housed in the base, wiring between the control unit and the detector must take into account movement of the imaging unit, and the configuration tends to become complicated. In contrast, when the detector is disposed in the imaging unit together with the control unit instead of the base, the configuration for wiring to electrically connect the detector and the control unit is easily simplified. Furthermore, the display unit may be attached to the imaging unit. As a result, the configuration for wiring to electrically connect the control unit and the display unit is also easily simplified.

In addition, the position of the detector within the imaging unit is below the control unit, which serves as a heat source, so even when the detector is disposed together with the control unit in the imaging unit, heat from the control unit hardly affects the detector. Thus, for example, when the detector is a spectrometer, variation in a total optical path length of the spectrometer that would otherwise reduce a signal-to-noise ratio of a signal is suppressed. The spectrometer includes a plurality of optical elements and a mount portion that holds the optical elements. Each of the optical elements includes at least a diffraction grating that disperses interference light of measurement light and reference light and a light-receiving element that detects the dispersed interference light.

Furthermore, since the detector is disposed in the imaging unit instead of the base, the base can be downsized and an installation area of the apparatus can be reduced. In particular, when the detector is a spectrometer, suppression of installation area can be expected.

The fundus imaging apparatus of the present embodiment includes a blowing mechanism for generating an air flow and an exhaust port for discharging air. Since air flows through the imaging unit by the blowing mechanism and is discharged outside the imaging unit from the exhaust port, air heated by the control unit can be efficiently discharged. When including the blowing mechanism and the exhaust port, by providing the blowing mechanism at a side surface of the control unit and the exhaust port at the same height as the control unit, circulation of air within the imaging unit is suppressed, and thermal changes in the imaging optical system and the OCT optical system can be more effectively suppressed.

In the fundus imaging apparatus of the present embodiment, since a plurality of blowing mechanisms are arranged in parallel on a side surface of the control unit, air heated by the control unit can be discharged more efficiently. For example, when the blowing mechanisms are arranged in parallel in a short-axis direction of the control unit on the side surface of the control unit, part of the blowing mechanisms are located above the control unit, and therefore not all air can be directed at the control unit. Accordingly, cooling efficiency deteriorates. In contrast, when the blowing mechanisms are arranged in parallel in a longitudinal direction of the control unit on the side surface of the control unit, the blowing mechanisms are disposed along the control unit, and therefore all blown air can be directed at the control unit. Accordingly, cooling efficiency is improved.

### Modified Example

The technology disclosed in the present embodiment is merely an example. Therefore, at least a part of the technology exemplified in the present embodiment can be modified.

In the present embodiment, as long as the control unit 100 is disposed above the imaging optical system 200, the control unit 100 may be placed at any position. That is, the control unit 100 may be disposed at any position in an XZ plane relative to a top surface of the enclosure 200A, and at any position in a Y direction. However, it is preferable that the control unit 100 be disposed so as not to protrude as much as possible from the top surface of the enclosure 200A, in consideration of slimming the width of the imaging unit 3.

In the present embodiment, the control unit 100 may also be disposed below the objective optical system 220. However, in this case, at least the objective optical system 220 is more likely to be affected by thermal changes due to rising heated air generated by the control unit 100.

1. A fundus imaging apparatus, comprising:
a base;
a display unit;
an imaging optical system configured to capture at least a color image as a fundus front image of a subject eye;
an OCT optical system configured to acquire OCT data of a fundus of the subject eye;
a control unit configured to execute at least (i) imaging control of the imaging optical system and the OCT optical system, (ii) display control processing of the color image and an OCT image based on the OCT data, and (iii) analysis processing of the OCT data; and
an imaging unit housing the imaging optical system, the OCT optical system, and the control unit therein, the imaging unit being disposed on the base, wherein
the control unit is disposed above the imaging optical system and the OCT optical system in the imaging unit,
the imaging unit includes an exhaust port that discharges air heated by the control unit, and
the exhaust port is located at the same height as the control unit or at a position higher than the control unit.

2. A fundus imaging apparatus, comprising:
a base;
a display unit;
an imaging optical system configured to capture at least a color image as a fundus front image of a subject eye;
an OCT optical system configured to acquire OCT data of a fundus of the subject eye;
a control unit configured to execute at least (i) imaging control of the imaging optical system and the OCT optical system, (ii) display control processing of the color image and an OCT image based on the OCT data, and (iii) analysis processing of the OCT data; and
an imaging unit housing the imaging optical system, the OCT optical system, and the control unit therein, the imaging unit being disposed on the base, wherein
the control unit is disposed above the imaging optical system and the OCT optical system in the imaging unit.

3. The fundus imaging apparatus according to claim 2, further comprising:
an optical path coupling member configured to combine an optical path of the imaging optical system and an optical path of the OCT optical system; and
an objective optical system disposed between the subject eye and the optical path coupling member to guide light to the fundus, wherein
the control unit is disposed above an optical axis of the objective optical system.

4. The fundus imaging apparatus according to claim 2, wherein
the control unit is disposed above an enclosure that covers the imaging optical system and the OCT optical system.

5. The fundus imaging apparatus according to claim 2, further comprising
a drive unit configured to move the imaging unit relative to the base, wherein
the OCT optical system includes a detector that detects interference light between reflection light of measurement light and reference light, and
the detector is disposed in the imaging unit.

6. The fundus imaging apparatus according to claim 2, wherein
the imaging unit includes an exhaust port for discharging air.

7. The fundus imaging apparatus according to claim 6, wherein
the exhaust port is disposed above the imaging unit.

8. The fundus imaging apparatus according to claim 6, further comprising
a blowing mechanism disposed in the imaging unit to face a side surface of the control unit to form an air flow, wherein
the air flows through the imaging unit by the blowing mechanism and is discharged outside the imaging unit through the exhaust port.

9. The fundus imaging apparatus according to claim 8, wherein
the blowing mechanism are a plurality of blowing mechanisms that are arranged in parallel to face the side surface of the control unit.
